(19) United States
(12) Patent Application Publication
(10) Pub. No.: US 2018/0372759 A1
(43) Pub. Date: Dec. 27, 2018

(54) DETERMINING ONSET OF A SLEEP-RELATED ANALYTE

(71) Applicant: KONINKLIJKE PHILIPS N.V., Eindhoven (NL)

(72) Inventors: ALEXANDER VAN REENEN, VUGHT (NL); MARKUS HENDRIKUS VAN ROOSMALEN, BERKEL-ENSCHOT (NL); LAURENT BROUQUEYRE, MARIETTA, GA (US); PETRUS JOHANNES WILHELMUS VAN LANKVELT, BOEKEL (NL)

(21) Appl. No.: 16/018,668

(22) Filed: Jun. 26, 2018

Related U.S. Application Data
(60) Provisional application No. 62/525,311, filed on Jun. 27, 2017.

(51) Int. Cl. G01N 33/74

(52) U.S. Cl. CPC G01N 33/74; G01J 1/00; G01N 2333/575

(57) ABSTRACT

Disclosed is a method for determining onset of a sleep-related analyte. The method includes: obtaining first surrogate measurement(s) of a sleep-related analyte based on signal(s) obtained via sensor(s) during a first time period of observation; obtaining second surrogate measurement(s) of the sleep-related analyte based on signal(s) obtained via sensor(s) during a second time period of observation; determining a first surrogate function or value based on the first surrogate measurement(s) and a second surrogate function or value based on the second surrogate measurement(s); and predicting an onset of the sleep-related analyte for a first individual based on the first surrogate function or value and the second surrogate function or value such that the prediction of the onset of the sleep-related analyte is performed without determining an absolute concentration value for the sleep-related analyte.

### CROSS-REFERENCE TO RELATED APPLICATIONS

This patent application claims the priority benefit under 35 U.S.C. § 119(e) of U.S. Provisional Application No. 62/525,311 filed on Jun. 27, 2017, the contents of which are herein incorporated by reference.

### BACKGROUND

### 1. Field

The present disclosure pertains to analyte onset detection systems and methods, including, for example, prediction of the onset of a sleep-related analyte for individuals.

### 2. Description of the Related Art

Analysis of the levels of a released substance or chemical constituent (e.g., glandular, hormonal, neurohormonal, etc.), including melatonin, adrenaline, insulin, aldosterone, antidiuretic hormone, oxytocin, prolactin, ghrelin, leptin, cortisone, cortisol, blood levels of sugar or cholesterol, or other substance/constituent, in an individual, may provide important and helpful prognostic information. For example, prediction of the melatonin secretion onset time at dim light conditions, i.e., the time at which the dim light melatonin onset (DLMO) occurs, is useful for analyzing or diagnosing various diseases and circadian disorders, such as circadian rhythm sleep disorders (e.g., insomnia), jet lag, seasonal affective disorder, shift work-related de-synchronies, and the delayed sleep phase syndrome. As a further example, the DLMO normally occurs in the evening (e.g., a few hours before sleep), but, in case of a circadian disorder, the DLMO can vary or be shifted.

Known methods for determining onset of a sleep-related analyte (e.g., the DLMO) require measuring the actual concentrations of the analyte in human samples of blood, saliva, urine, or other bodily fluid. To accurately measure such concentrations, known methods require costly calibration systems to calibrate the concentration measurement tools. Moreover, because the actual concentrations of the analyte in the samples are compared against a predefined concentration level to determine the analyte onset, the onset determination is susceptible to variations between different donors. Specifically, for example, the concentration of melatonin that corresponds to DLMO may vary among individuals, and, as such, using the same predefined concentration level (as a threshold comparison) often leads to errors in the DLMO time measurement.

In addition, known methods include taking a predefined number of samples from an individual, each of the samples being taken at a different time interval, and sending the samples to a laboratory for analysis for analyte onset determination, thereby often resulting in one or more weeks of delay with respect to determining the onset of the analyte. Further, to ensure that a sufficient number of samples are sent for analysis, the predefined number of samples taken is often greater than the actual number of samples that are needed to determine the analyte onset, thereby resulting in greater inconvenience to the individual. In the case of DLMO, for example, the individual is caused to stay awake longer than needed or risk having to repeat the entire procedure. These and other drawbacks exist.

### SUMMARY

Accordingly, one or more aspects of the present disclosure relate to a system configured to determining an analyte's onset. The system comprises one or more hardware processors and/or other components. In some embodiments, the one or more hardware processors are configured by machine-readable instructions to: obtain one or more first surrogate measurements of a sleep-related analyte based on one or more signals obtained via one or more sensors during a first time period of observation of a first individual; obtain one or more second surrogate measurements of the sleep-related analyte based on one or more signals obtained via one or more sensors during a second time period of observation of the first individual; determine a first surrogate function or value based on the one or more first surrogate measurements and a second surrogate function or value based on the one or more second surrogate measurements; and predict an onset of the sleep-related analyte for the first individual based on the first surrogate function or value and the second surrogate function or value such that the prediction of the onset of the sleep-related analyte is performed without the one or more processors determining an absolute concentration value for the sleep-related analyte.

Yet another aspect of the present disclosure relates to a method for determining an analyte's onset. The method is implemented by one or more processors configured by machine-readable instructions and/or other components. The method comprises: obtaining one or more first surrogate measurements of a sleep-related analyte based on one or more signals obtained via one or more sensors during a first time period of observation of a first individual; obtaining one or more second surrogate measurements of the sleep-related analyte based on one or more signals obtained via one or more sensors during a second time period of observation of the first individual; determining a first surrogate function or value based on the one or more first surrogate measurements and a second surrogate function or value based on the one or more second surrogate measurements; and predicting an onset of the sleep-related analyte for the first individual based on the first surrogate function or value and the second surrogate function or value such that the prediction of the onset of the sleep-related analyte is performed without the one or more processors determining an absolute concentration value for the sleep-related analyte.

Still another aspect of the present disclosure relates to a system for determining an analyte's onset. The system comprises: means for obtaining one or more first surrogate measurements of a sleep-related analyte based on one or more signals obtained via one or more sensors during a first time period of observation of a first individual; means for obtaining one or more second surrogate measurements of the sleep-related analyte based on one or more signals obtained via one or more sensors during a second time period of observation of the first individual; means for determining a first surrogate function or value based on the one or more first surrogate measurements and a second surrogate function or value based on the one or more second surrogate measurements; and means for predicting an onset of the sleep-related analyte for the first individual based on the first surrogate function or value and the second surrogate function or value such that the prediction of the onset of the sleep-related analyte is performed without determining an absolute concentration value for the sleep-related analyte.

These and other objects, features, and characteristics of the present disclosure, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosure.

### BRIEF DESCRIPTION OF THE DRAWINGS

FIG. 1 is a schematic illustration of a system configured to determine an analyte's onset, in accordance with one or more embodiments;

FIG. 2 illustrates an example of a competitive inhibition immunoassay format, in accordance with one or more embodiments;

FIG. 3 illustrates an exemplary in-vitro diagnostics device embeddable in a measurement tool, in accordance with one or more embodiments;

FIG. 4 illustrates a melatonin dose-response inhibition curve, in accordance with one or more embodiments;

FIG. 5 illustrates an exemplary signal profile of normalized data points that relate to an analyte's concentrations, in accordance with one or more embodiments;

FIG. 6 illustrates an exemplary signal profile of normalized data points that relate to an analyte's concentrations, in accordance with one or more embodiments;

FIG. 7 illustrates a method for determining an analyte's onset, in accordance with one or more embodiments;

FIG. 8 illustrates a method for determining an analyte's onset in minimal time, in accordance with one or more embodiments;

FIG. 9 illustrates a method for determining an analyte's onset using a dynamic threshold, in accordance with one or more embodiments; and

FIG. 10 illustrates a method for determining an analyte's onset using an adapted hockey-stick algorithm, in accordance with one or more embodiments.

### DETAILED DESCRIPTION OF EXEMPLARY EMBODIMENTS

As used herein, the singular form of “a”, “an”, and “the” include plural references unless the context clearly dictates otherwise. As used herein, the term “or” means “and/or” unless the context clearly dictates otherwise. As used herein, the statement that two or more parts or components are “coupled” shall mean that the parts are joined or operate together, either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example, and without limitation, top, bottom, left, right, upper, lower, front, back, and derivatives thereof, relate to the orientation of the elements shown in the drawings and are not limiting upon the claims unless expressly recited therein.

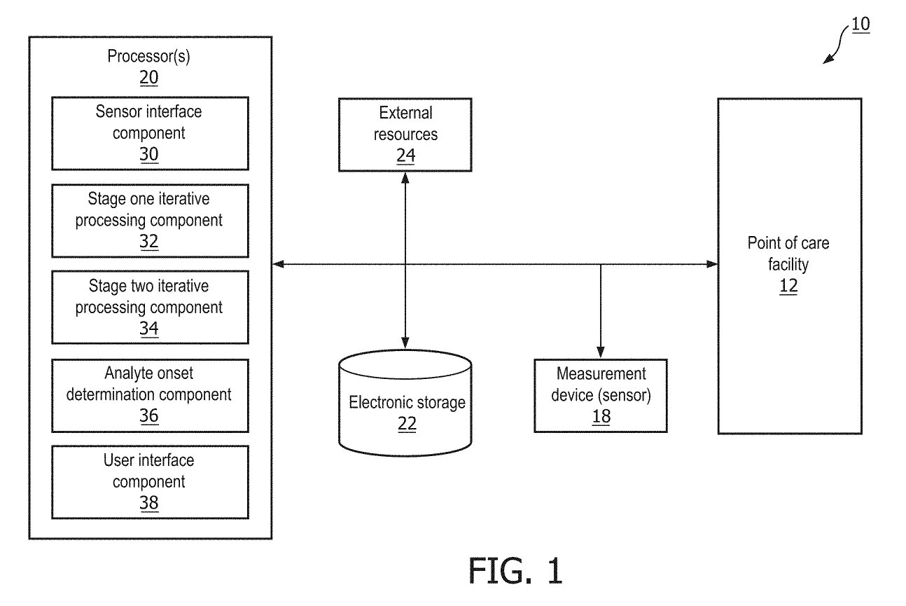

FIG. 1 illustrates a system 10 configured to determine an analyte's onset. In some embodiments, system 10 may be configured to determine onset in real-time of an analyte (e.g., a sleep-related analyte, such as melatonin, or other analyte) by at least measuring a plurality of samples, which are taken from an individual in one or more time periods of observation. In some embodiments, measurements of an analyte, including physiological, metabolized products thereof, are each a surrogate measurement. Such measurements are surrogates for calculating analyte concentrations, i.e., being merely related to the concentration. That is, rather than determining an absolute concentration value of an analyte (e.g., in terms of pico-grams (pg) per milliliter (mL)), a surrogate measurement is taken for each analyte sample from the individual, and relative changes in the surrogate measurement of the analyte may be analyzed for determining an onset on the analyte. Such relative displacements between data points or across a data signal profile render this approach independent of variations between individuals.

In some embodiments, system 10 is configured to determine the onset of an analyte for an individual during observation of the individual. In some embodiments, rather than require the individual to undergo giving a predetermined number of samples, system 10 may determine the analyte's onset based on a number of samples from the individual that is less than the predetermined number of samples. In some embodiments, system 10 may indicate that no further samples are needed from the individual, responsive to a determination that the onset of the analyte has occurred. As an example, when system 10 predicts that the onset of the analyte has occurred, and such prediction satisfies a threshold level of accuracy, system 10 may transmit an indication that no further samples are needed from the individual. In one use case, the transmission of the indication may include transmission of one or more instructions to one or more components of system 10 to stop further obtainment of samples from the individual, thereby causing the obtainment of samples from the individual to end. In this way, for example, the individual need not wait until the predetermined number of samples are taken, thereby reducing the amount of time that the individual needs to remain under observation for sampling purposes and/or the amount of time for determining the analyte's onset.

In some embodiments, system 10 is configured to determine the onset with an increased probability in comparison to a diagnostic approach where a predetermined set of samples is taken. That is, in a scenario where the set of samples is not enough (e.g., the analyte onset has not yet happened after the entire set is taken), then system 10 may prolong the sample-taking period until the onset is determined. In other words, system 10 may adjust the sample taking period to allow for more measurement samples to be taken to increase the probability that a determination of the onset of the analyte occurs.

In some embodiments, system 10 is configured to analyze the complete data-set of measurement samples obtained, including the most recent measurement, in determining whether an onset has occurred. In these or other embodiments, two samples may be enough for determining the onset. The operations performed by system 10 may be performed at different times. For example, system 10 may take and receive measurements and carry out the operations described herein at any time of day and for any duration of time, but preferably in relation to a time when an analyte is known to be released in the body. The onset of melatonin secretion in the evening, for example, is very reliable.

The description and illustration herein (FIG. 1) of a single point of care facility 12 is not intended to be limiting. Point of care facility 12 may represent any number of point of care facilities, such as a health care facility (e.g., a hospital, hospital system, clinic, doctor's office, laboratory, or other health care facility), an individual's home, or other point of care facility. The operations performed by system 10 are applied individually to any number of facilities 12.

In some embodiments, system 10 further comprises one or more measurement devices 18, one or more processors 20, electronic storage 22, external resources 24, and/or other components. Measurement devices 18 may be based on a diagnostic platform that includes an interface between users' fluidic samples of an assay and system 10, when taking measurements. In some embodiments, measurement devices 18 are point of care devices (e.g., a device similar to the Minicare device by PHILIPS), and they may be associated with a point of care facility 12 and/or other users, service providers, and/or entities participating in point of care facility 12. Service providers may be nurses, paramedics, caregivers, medical consultants, or any provider of service associated with a point of care facility. Measurement devices 18 are configured to provide information to and/or receive information from such users and/or entities. Measurement device 18 (i.e., the diagnostics platform) may include an analyzer device component and a disposable cartridge component. The analyzer component may include detection mechanisms and magnetic actuation mechanisms to perform a measurement, and the cartridge component may perform the biological functions for the measurement.

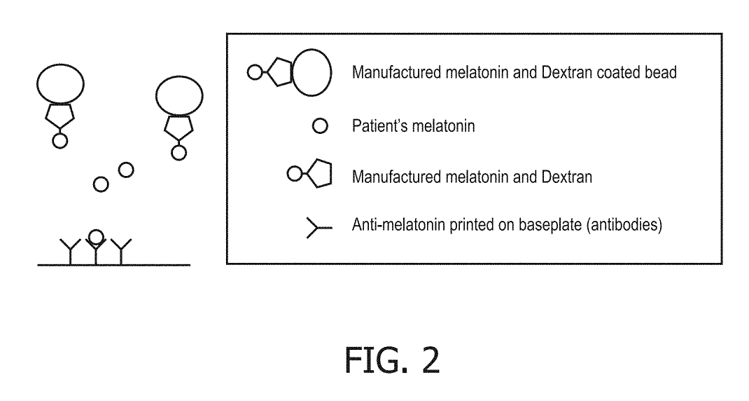

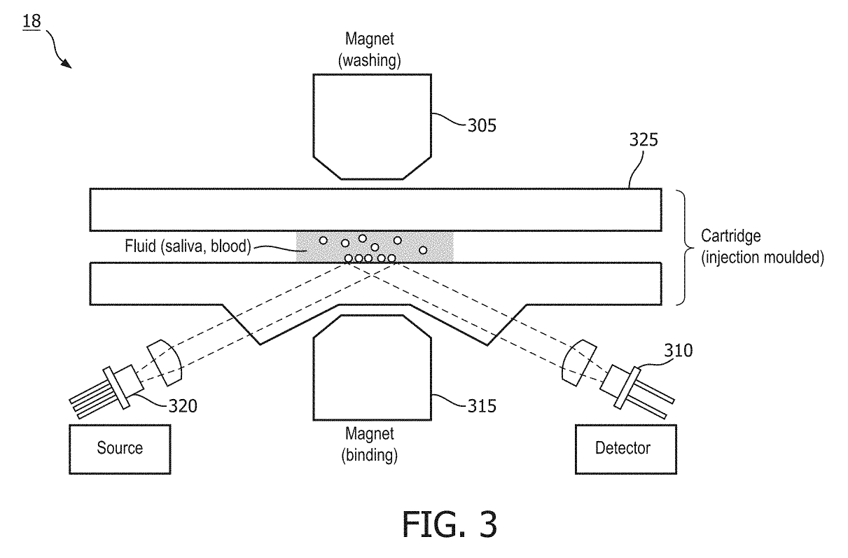

In some embodiments, measurement device 18 is configured to perform an immunoassay using magnetic or paramagnetic beads that react with an analyte of samples that are input to a cartridge of the device. Such assay is discussed below, with reference to FIGS. 2-3. In some embodiments, measurement device 18 is configured with respect to another type of assay or technique to measure levels of the analyte, such as enzyme-linked immunosorbent assays (ELISA) or liquid chromatography-mass spectroscopy (LC-MS). ELISA is an analytic biochemistry assay that uses a solid-phase enzyme immunoassay (EIA) to detect the presence of a substance. LC-MS is an analytical chemistry technique that combines the physical separation capabilities of liquid chromatography with the mass analysis capabilities of mass spectrometry. The type of assay presented herein is not intended to be limiting, as any suitable technique that provides a surrogate measurement may be used, the measurement being a surrogate for determining an absolute analyte concentration.

In some embodiments, one or more measurement devices 18 are configured to provide a user interface and/or other inputs/outputs (I/O) interface, processing capabilities, databases, and/or electronic storage to system 10. As such, measurement devices 18 may include one or more sensors, a processor, storage, and/or other components. The sensors may include one or more light sensors (e.g., photoresistor-based sensors, photodiode-based sensors, phototransistor-based sensors, etc.), sound sensors (e.g., microphones), chemical sensors (e.g., sensors that include a chemical (molecular) recognition system (receptor), a physicochemical transducer, etc.), or other sensors.

Processor 20 is configured to provide information processing capabilities in system 10. As such, processor 20 may comprise one or more of a digital processor, an analog processor, a digital circuit designed to process information, an analog circuit designed to process information, a state machine, and/or other mechanisms for electronically processing information. Although processor 20 is shown in FIG. 1 as a single entity, this is for illustrative purposes only. In some embodiments, processor 20 may comprise a plurality of processing units. These processing units may be physically located within the same device (e.g., a server), or processor 20 may represent processing functionality of a plurality of devices operating in coordination (e.g., one or more servers, measurement devices 18, devices that are part of external resources 24, electronic storage 22, and/or other devices.) In some embodiments, processor(s) 20 are embedded in a server, laptop, desktop computer, smartphone, tablet computer, smart watch, and/or another computing device.

In some embodiments, processors 20, external resources 24, measurement devices 18, electronic storage 22, systems that are part of point of care facility 12, and/or other components may be operatively linked via one or more electronic communication links. For example, such electronic communication links may be established, at least in part, via a network such as the Internet, and/or other networks. It will be appreciated that this is not intended to be limiting, and that the scope of this disclosure includes embodiments in which these components may be operatively linked via some other communication media. In some embodiments, processor 20 is configured to communicate with external resources 24, measurement devices 18, electronic storage 22, the systems that are part of point of care facility 12, and/or other components according to a client/server architecture, a peer-to-peer architecture, and/or other architectures.

As shown in FIG. 1, processor 20 is configured via machine-readable instructions to execute one or more computer program components. The computer program components may comprise one or more of sensor interface component 30, stage-one iterative processing component 32, stage-two iterative processing component 34, analyte onset determination component 36, user interface component 38, and/or other components. Processor 20 may be configured to execute components 30, 32, 34, 36 and/or 38 by: software; hardware; firmware; some combination of software, hardware, and/or firmware; and/or other mechanisms for configuring processing capabilities on processor 20.

It should be appreciated that although components 30, 32, 34, 36, and 38 are illustrated in FIG. 1 as being co-located within a single processing unit, in embodiments in which processor 20 comprises multiple processing units, one or more of components 30, 32, 34, 36, and/or 38 may be located remotely from the other components. The description of the functionality provided by the different components 30, 32, 34, 36, and/or 38 described below is for illustrative purposes, and is not intended to be limiting, as any of components 30, 32, 34, 36 and/or 38 may provide more or less functionality than is described. For example, one or more of components 30, 32, 34, 36, and/or 38 may be eliminated, and some or all of its functionality may be provided by other components 30, 32, 34, 36, and/or 38. As another example, processor 20 may be configured to execute one or more additional components that may perform some or all of the functionality attributed below to one of components 30, 32, 34, 36, and/or 38.

In some embodiments, sensor interface component 30 is configured to receive from measurement device 18 one or more output signals from one or more of its sensors (or other type of detector of a source signal), which convey a measurement for an individual at point of care facility 12. In some embodiments, sensor interface component 30 is configured to obtain measurements with regard to human samples taken from serum (e.g., blood or plasma), saliva, urine, and/or another bodily fluid.

In some embodiments, sensor interface component 30 may support reception of any scale of the received signal, this feature being necessary since the measurement tool may be uncalibrated and since each individual (and across a plurality of individuals) on any given night or at any point in time may produce samples with markedly different analyte measurement levels. In some embodiments, the measurements obtained by sensor interface component 30 may be obtained without determining absolute analyte concentrations.

In some embodiments, stage-one iterative processing component 32 and stage-two iterative processing component 34 are configured to process the measurements obtained by sensor interface component 30 in first and second stages of observation, respectively. In some embodiments, stage-one iterative processing component 32 and stage-two iterative processing component 34 are each configured to determine a surrogate function (referred to herein merely as a “function”). For example, the surrogate functions may each be a polynomial function, a collection or plot of points, a signal profile, or other relationship between inputs and outputs, which may include the plurality of measurements of the analyte taken in the respective stage of observation.

In some embodiments, stage-one iterative processing component 32 is configured to determine a baseline portion of the measurements in the first stage, e.g., a first time period of observation. The baseline portion may be determined for a time period when analyte onset has not yet occurred, e.g., by restricting a determined function representing the obtained measurements to have a particular range for its slope. In some implementations, the restricted range of the slope may be between negative 0.2 and positive 0.2. This baseline portion may be referred to as the flat or constant portion. A flat portion may have a zero or negligible slope, and a constant portion may have a constant (e.g., linear) slope. In some instances, the first stage may be characterized by a series of measurements that have neither a flat nor constant slope but that still represents a period of time where the analyte onset has not yet occurred. In some embodiments, stage-one iterative processing component 32 is configured to determine the baseline portion by determining a function or value based on the measurements taken in the first time period and comparing the determined function or value with another value or function. For example, an average, median, or mode of the measurements taken may be determined to represent some or all of the measurements taken in the first time period.

In some embodiments, stage-two iterative processing component 34 is configured to determine a function or value based on the measurements taken in the second stage, e.g., a second time period of observation. In some embodiments, stage-two iterative processing component 34 is configured to calculate a function or value based on these measurements such that the determined function or value is compared with another value or function. For example, stage-two iterative processing component 34 may determine a threshold value against which the determined value may be compared. In another example, stage-two iterative processing component 34 may determine lines or curves against which the determined function may be fitted.

In some embodiments, stage-two iterative processing component 34 is configured to cooperate with stage-one iterative processing component 32. For example, functions representing the measured analyte in both stages may, by a piecewise linear-parabolic function, be represented as a straight line switching to the branch of a parabola. In this or another example, the hockey-stick algorithm (see, e.g., “The Hockey-Stick Method to Estimate Evening Dim Light Melatonin Onset (DLMO) in Humans” by Danilenko et al.; Chronobiology International Journal, Early Online: 1-7, (2013)) may be used as a reliable objective method to estimate when an analyte's measurement ramps up or down, such algorithm being potentially independent of threshold values and free from errors arising from differences in subjective estimates.

In some embodiments, analyte onset determination component 36 is configured to reliably and objectively determine an analyte's onset, e.g., at a transition between the first and second stages. For example, stage-two iterative processing component 34 may determine the onset either by a fixed or dynamic threshold. In another example, stage-two iterative processing component 34 is configured to reliably and objectively determine the analyte's onset using a curve-fitting method (e.g., based on an adapted hockey-stick algorithm or a method based on the hockey-stick algorithm). That is, by measuring samples at multiple time-points, the one or more measurements in the first and/or second time periods may be analyzed or a signal profile may be obtained from the respective measurements for analysis.

In some embodiments, analyte onset determination component 36 is configured to calculate the analyte's onset, after the surrogate assay, from the serially taken samples by linear interpolation between adjacent points, e.g., by looking for the time at which ramping analyte levels cross a predetermined threshold. This is merely one example for determining the onset. That is, analyte onset determination component 36 may be configured to determine the onset using any suitable method. Some methods that may be used to determine the onset include visual inspection (e.g., subjectively, by an expert, or via a sleep log or polysomnography), threshold-based, curve-fitting, and differential equation-based methods.

In some embodiments, analyte onset determination component 36 is configured to support early analyte onset detection, e.g., if a clear ramp or bend away from the baseline portion is observed in the measurements. In some use cases, no significant ramp is detectable in the measurements of the first or second stage, e.g., when failing to determine the onset.

In some embodiments, analyte onset determination component 36 may use a threshold-based method. Threshold-based methods use either a predetermined threshold (e.g., around 3 to 4 pg/mL for a saliva sample with melatonin and 10 pg/mL for a blood/plasma sample with melatonin) or a dynamic threshold value that relates to the initial measurement data (e.g., double a standard deviation). To determine the onset, the best methods make use of interpolation techniques (e.g., linear, cubic, or other) to accurately estimate at what time the analyte level crossed the threshold. Threshold-based methods are easy to apply, but they may overestimate the onset and may not process any information from the shape of the measured profile; overestimation of an onset may be due to expectation of a higher analyte measurement at a later period in time than what is appropriate for characterizing the onset. Measurement profiles, e.g., may show a weak or strong rise after the onset and, when compared against a threshold, lead to temporal deviations from the onset. Threshold-based methods may, in some embodiments, not account for different profile shapes and a steepness of the serial measurements.

In some embodiments, analyte onset determination component 36 may use a curve-fitting method. Curve-fitting methods may objectively use predetermined fit functions to determine the onset, i.e., comparable against a variety of different signal profiles. For example, local polynomial fitting may be applied to fit statistical data over a time period, such as 24 hours. Local polynomial fitting may determine that a first peak in a calculated derivative is the time at which analyte production levels were increased. One known method, which is suitable for sparse datasets containing only measurements taken in an evening, is the hockey-stick method. This method may apply or fit a linear function (e.g., with slopes restricted to a predetermined value such as +/−2 pg/mL in a certain time period) to fit the data at smaller analyte levels where flat or constant signals are obtained; the method may also apply or fit a polynomial (e.g., parabolic) function to the data where there are rising analyte levels. The onset can thus be predicted based on a point of connection or switching from the linear function to the polynomial function. Several constraints and conditions may be used to ensure a reliable prediction, effectively making curve-fitting based methods more complex than visual inspection and threshold-based methods.

In some embodiments, analyte onset determination component 36 may use a differential equation-based method. Differential equation-based methods are basically fitting methods, but they more explicitly describe the physiological pathway of the analyte regulation in the human body. They start from assuming a mathematical function describing the activity of the gland that produce more or less of the analyte, which is inserted in a system of differential equations describing the analyte level in the gland (lowered by excretion of the analyte into the blood stream), as well as the analyte level in the blood plasma, where the analyte enters from the gland and is cleared. Using this mathematical model, 24-hourly data may be fitted to extract the rate and time of increased and decreased analyte synthesis (e.g. the onset and offset time). Although these methods may provide many useful insights, they may not reliably be used on infrequently-taken, inaccurate, or sparse datasets containing only measurements taken, e.g., in the evening.

In some embodiments, analyte onset determination component 36 is configured to obtain high-levels of precision with respect to the analyte's onset using a dynamic threshold or the adapted hockey-stick method. In some embodiments, analyte onset determination component 36 is configured to support relatively large variations in the determined onset time, e.g., between different individuals or between different observation periods for the same individual.

In some embodiments, analyte onset determination component 36 is configured to determine the onset in less than fifteen minutes, thirty minutes, forty-five minutes, one hour, etc. In these embodiments, the time-to-analyze may be less than a sampling interval in either the first or second time period. For example, the sampling interval may be hourly, and analyte onset determination component 36 may be able to determine the onset in less than sixty minutes, i.e., from the interval at which the most recent measurement is taken.

In some embodiments, analyte onset determination component 36 is configured to determine the onset in optimal time. For example, in the case of determining the DLMO, a subject may be able to go home sooner, rather than waiting to undergo a complete set of measurements in dim light conditions for several hours. In some instances, though, an analyte onset may not yet have been detected and thus more measurements may be needed beyond the complete set. For example, to avoid a subject undergoing another set of measurements, a user could take a seventh or eighth analyte measurement if the complete set is limited to six measurements.

In some embodiments, analyte onset determination component 36 is configured to determine an analyte's offset (e.g., when the measured signal is ramping in a different direction than the onset), which may be some time period after the determined onset. For example, rather than merely focusing on changes in measurements from the flat or constant portion, system 10 may take additional measurements (or only take these measurements, in some embodiments) to focus on changes in measurements after analyte saturation in returning to a flat or constant portion. That is, the offset determination may be based on fitting a steep bend or curve to the measurements or based on the measurement(s) crossing a threshold just before returning to the flat or constant portion.

In some embodiments, user interface component 38 is configured to receive user-input, e.g., for setting threshold values against which analyte onset determination component 36 compares functions or values. That is, the function or value determined by stage-one iterative processing component 32 and/or stage-two iterative processing component 34 may be compared against user-configurable functions or values. Examples of interface devices suitable for inclusion in the user interface of system 10 include a touch screen, a keypad, touch sensitive and/or physical buttons, switches, a keyboard, knobs, levers, a display, speakers, a microphone, an indicator light, an audible alarm, a printer, and/or other interface devices. The present disclosure also contemplates that user interface component 38 may interface with a removable storage interface. In this example, information may be loaded into processor(s) 20 from removable storage (e.g., a smart card, a flash drive, a removable disk) that enables users to customize its implementation. Other exemplary input devices and techniques adapted for use with the user interface include, but are not limited to, an RS-232 port, RF link, an IR link, a modem (telephone, cable, etc.) and/or other devices. In some embodiments, user interface component 38 interfaces with measurement device 18, e.g., for obtaining taken measurements.

Returning to FIG. 1, electronic storage 22 comprises electronic storage media that electronically stores information. The electronic storage media of electronic storage 22 may comprise one or both of system storage that is provided integrally (i.e., substantially non-removable) with system 10 and/or removable storage that is removably connectable to system 10 via, for example, a port (e.g., a USB port, a firewire port, etc.) or a drive (e.g., a disk drive, etc.). Electronic storage 22 may be (in whole or in part) a separate component within system 10, or electronic storage 22 may be provided (in whole or in part) integrally with one or more other components of system 10 (e.g., a measurement device 18, processor 20, etc.). In some embodiments, electronic storage 22 may be located in a server together with processor 20, in a server that is part of external resources 24, in measurement devices 18, and/or in other locations. Electronic storage 22 may comprise one or more of optically readable storage media (e.g., optical disks, etc.), magnetically readable storage media (e.g., magnetic tape, magnetic hard drive, floppy drive, etc.), electrical charge-based storage media (e.g., EPROM, RAM, etc.), solid-state storage media (e.g., flash drive, etc.), and/or other electronically readable storage media.

Electronic storage 22 may store software algorithms, information obtained and/or determined by processor 20, information received via measurement devices 18 and/or other external computing systems, information received from external resources 24, information received from point of care facility 12, and/or other information that enables system 10 to function as described herein. By way of a non-limiting example, electronic storage 22 may store the surrogate measurements obtained by sensor interface component 30 or directly from measurement device 18, a time at which surrogate measurements are taken, the baseline value determined by stage-one iterative processing component 32, the thresholds determined by stage-one and stage-two iterative processing components 32 and 34, the time of onset determined by analyte onset determination component 36, and/or other information.

External resources 24 include sources of information (e.g., databases, websites, etc.), external entities participating with system 10 (e.g., a medical records system of a point of care facility that stores patient information), one or more servers outside of system 10, a network (e.g., the Internet), electronic storage, equipment related to Wi-Fi technology, equipment related to Bluetooth® technology, data entry devices, and/or other resources. In some implementations, some or all of the functionality attributed herein to external resources 24 may be provided by resources included in system 10. External resources 24 may be configured to communicate with processor 20, measurement device 18, electronic storage 22, point of care facility 12, and/or other components of system 10 via wired and/or wireless connections, via a network (e.g., a local area network and/or the internet), via cellular technology, via Wi-Fi technology, and/or via other resources.

FIG. 2 is an example of a competitive inhibition immunoassay format. An immunoassay is any method for detecting a substance by using an antibody reactive with it. For example, anti-melatonin may be printed on a baseplate, as shown in FIG. 2. Such format may be used in an analysis for detecting analyte levels using magnetic or paramagnetic beads. In this example, melatonin is the analyte but any other sleep-related analyte or non-sleep-related analyte may be used in the assay. In such melatonin assay, free melatonin in the sample solution of the individual is detected in the presence of manufactured melatonin coupled to the beads. In this example, melatonin from the individual's taken sample binds to the sensor surface containing anti-melatonin antibodies. Subsequently, magnetic particles coated with melatonin (not from the sample, e.g., manufactured) are brought in contact with the surface. Depending on the amount of melatonin from the sample that has bound to the surface, more or less magnetic particles will bind to the surface. In other words, melatonin from the sample inhibits the binding reaction of magnetic particles to the surface.

FIG. 3 is an in-vitro diagnostics device embeddable in measurement device 18. Measurement device 18 may include magnets 305 and 315 for washing and binding, respectively, the manufactured analyte and dextran coated bead. That is, in one or more alternating phases, the magnets may cause the paramagnetic beads to move towards the baseplate when binding and move away from the baseplate when washing. A source of energy (e.g., laser, visible light, infrared, heat, or other form of radiation) from source 320 may then be reflected off the baseplate, where some of the sampled analyte is bound, such that the reflected energy is collected at detector 310. These operations may occur within cartridge 325. In some embodiments, the cartridge may be injection molded. FIG. 3 depicts just one manner in which a surrogate measurement of an analyte may be obtained without determining absolute analyte concentrations. For example, other in-vitro assays may be performed or any suitable in-vivo assay may be performed as part of the measurement-taking operations of system 10.

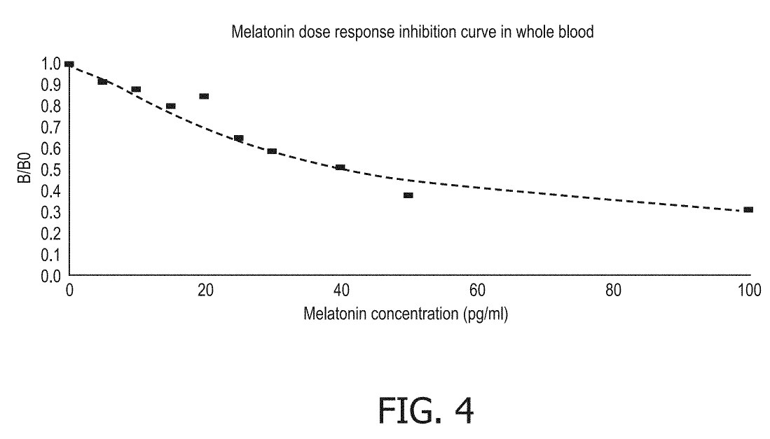

FIG. 4 illustrates a melatonin dose-response inhibition curve. On the X-axis is an equivalent melatonin concentration for a normalized signal (i.e., normalized with respect to a baseline melatonin level before the DLMO). The normalized signals are plotted with respect to the Y-axis. FIG. 4 depicts measured signals (e.g., from the magnetic bead-based melatonin assay) plotted as a function of different melatonin concentrations. Herein, B represents the measured signal, and B0 represents the signal corresponding to a baseline portion (e.g., where there is little or no melatonin in the measured sample(s)). B/B0 thus corresponds to the normalized signal. The plotted result shows that the assay is sensitive to melatonin concentrations.

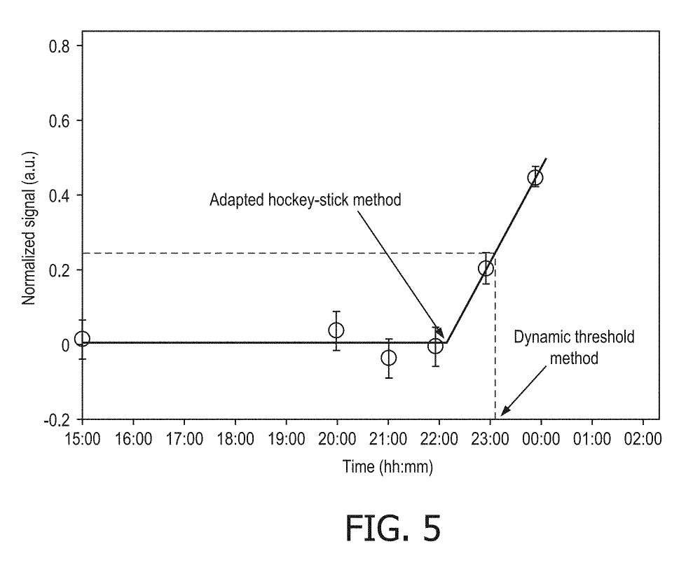

FIG. 5 illustrates a signal plot (or profile) of normalized data points that relate to an analyte's concentrations. On the X-axis is time, e.g., the time at which the particular measurements are taken. In this example, a measurement exists for 15:00 hours, 20:00 hours, 21:00 hours, 22:00 hours, 23:00 hours, and midnight. A first measurement, e.g., at 15:00 hours, may be a pseudo-measurement inserted based on predetermined baseline data. For example, the predetermined baseline data may be based on any suitable parameter such as a precision of measurement device 18, i.e., when no or a small amount of the analyte is present. In this way, an analyte's onset can still be determined with very limited baseline portion measurements. On the Y-axis is the normalized signal with respect to an arbitrary unit (a.u.). The normalized values may be based on measurements taken for an individual to determine an analyte signal profile. Based on the signal profile, the analyte's onset can then be determined without computing the analyte's absolute concentration values. As shown in FIG. 5, different methods may be applied to determine the onset time from the signal profile. In particular, the onset may be determined by using a dynamic threshold or an adapted version of the hockey-stick method.

In FIG. 5, the circles indicate the normalized, measured signals. Data is normalized using the conversion equation S=1+B/B0. The B0 baseline value may be predetermined or dynamically determined by analyzing a flat or constant portion of the signal profile and by using the measurements' average as an estimate for the B0. The solid curve of FIG. 5 may be a fit to the data points using, e.g., the adapted hockey-stick method. In the example provided in FIG. 5 and where the adapted hockey-stick method is used, the analyte's onset is determined to be 22:07 hours. Using the same data points of this example but where the dynamic threshold approach is used (represented with a dotted-line), the analyte's onset is determined to be 23:10 hours. In the latter case, the value of the threshold may be determined from an analysis of the flat or constant portion of the signal profile. That is, the dynamic threshold may, in some embodiments, be based on the standard deviation and/or an average of the signals measured in the first time period to form the flat or constant portion. For example, the dynamic threshold used may be beyond a baseline value by an amount equal to, e.g., the standard deviation, double the standard deviation, triple the standard deviation, quadruple the standard deviation, quintuple the standard deviation, any multiple of it, or another parameter or equation. In these examples, the dynamic threshold may be plotted (e.g., for visual inspection purposes) in relation to (e.g., above) the baseline portion.

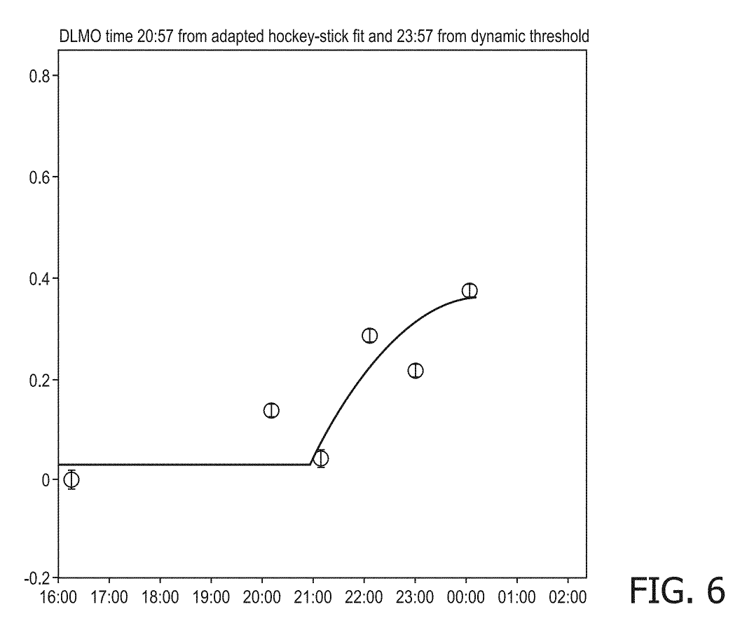

As in FIG. 5, FIG. 6 illustrates a signal plot (or profile) of normalized data points that relate to an analyte's concentrations. Here a polynomial shape, specifically a parabolic one, is discernible from the taken measurements. That is, FIG. 6 illustrates that the measured level of an analyte may be flat or constant in a first time period of observation and then ramp up in a second time period before saturating, which represents a typical melatonin excretion profile for an individual in an evening.

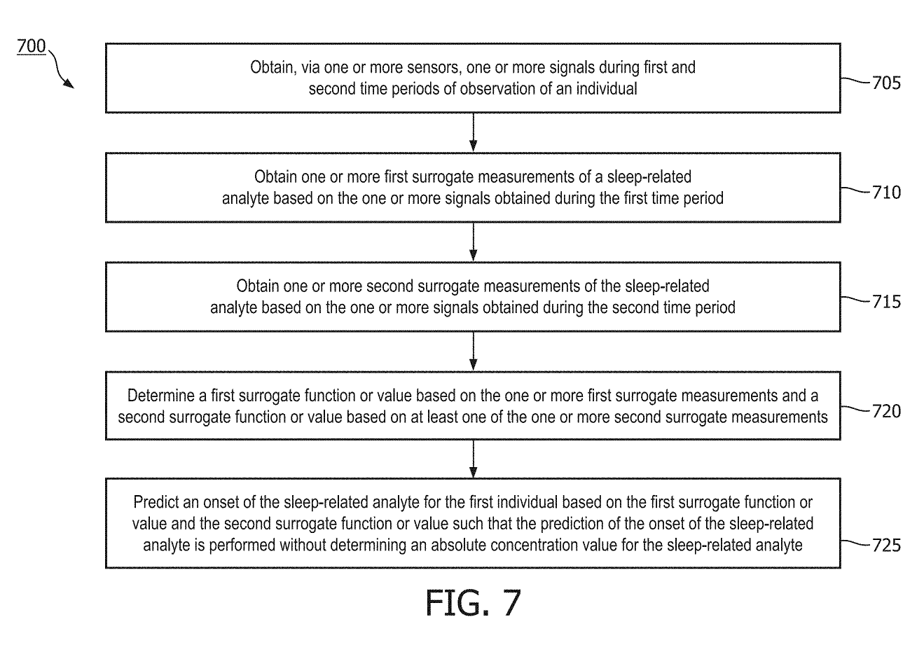

FIG. 7 illustrates a method 700 for determining an analyte's onset. Method 700 may be performed with a personnel scheduling system (e.g., system 10). The system comprises of one or more hardware processors and/or other components. The hardware processors are configured by machine-readable instructions to execute computer program components. The computer program components include a sensor interface component, stage-one iterative processing component, stage-two iterative processing component, analyte onset determination component, user interface component, and/or other components. The operations of method 700 presented below are intended to be illustrative. In some embodiments, method 700 may be accomplished with one or more additional operations not described, and/or without one or more of the operations discussed. Additionally, the order in which the operations of method 700 are illustrated in FIG. 3 and described below is not intended to be limiting.

In some embodiments, method 700 may be implemented in one or more processing devices (e.g., a digital processor, an analog processor, a digital circuit designed to process information, an analog circuit designed to process information, a state machine, and/or other mechanisms for electronically processing information). The processing devices may include one or more devices executing some or all of the operations of method 700 in response to instructions stored electronically on an electronic storage medium. The processing devices may include one or more devices configured through hardware, firmware, and/or software to be specifically designed for execution of one or more of the operations of method 700.

At an operation 705, the system may obtain, via one or more sensors, one or more signals during first and second time periods of observation of an individual. As an example, the system may interface directly or indirectly with the one or more sensors of a measurement device (e.g., measurement device 18). The one or more signals may be raw outputs based on detected energy levels. The detected energy may vary in accordance with an amount of an analyte found in a fluidic sample taken of an individual to which the energy is directed and against which the energy is reflected back into the one or more sensors (e.g., detector 310). In some use cases, the measurement device used for obtaining the first and second surrogate measurements may be uncalibrated. In some embodiments, operation 705 is performed by a processor component that is the same as or similar to sensor interface component 30 (shown in FIG. 1 and described herein).

At an operation 710, the system may obtain one or more first surrogate measurements of a sleep-related analyte based on the one or more signals obtained during the first time period. As an example, the first measurements may be an amount of energy reflected, e.g., into detector (sensor) 310, upon completion of binding and washing phases. These phases may take place via use of magnets 305 and 315, manufactured beads coated with dextran (the use of dextran is not intended to be limiting) and an analyte, the analyte of a bodily fluid sample, and anti-analytes (e.g., antibodies) printed on a baseplate of a cartridge in which the assay is performed. Such an assay may be similar to or the same as the one previously described with reference to FIGS. 2-3, i.e., each of the first and second surrogate measurements may be obtained via an immunoassay based on magnetic colloids or beads. For example, the signal received with respect to an initial measurement may indicate that 80% of the energy output from energy source 320 is reflected back into the detector. A subsequent signal, received some time later, may indicate a measurement of 60% reflection.

For each surrogate measurement described in this disclosure (e.g., with regard to operation 710), a relative or absolute time when that measurement was taken may be recorded or stored in a memory device (e.g., electronic storage 22). A number of times the one or more first surrogate measurements are taken increments until a beginning of a second time period is detected. In some embodiments, operation 710 is performed by a processor component the same as or similar to stage-one iterative processing component 32 (shown in FIG. 1 and described herein).

In some implementations, outlier (e.g., unexpectedly too large or small) data points (measurements) may be removed and/or replaced as part of this operation. Any outliers may be removed from measurements of the first and/or second time period.

At an operation 715, the system may obtain one or more second surrogate measurements of the sleep-related analyte based on the one or more signals obtained during the second time period. These measurements may be taken using the same or similar approach described with reference to operation 710. A number of times the one or more second surrogate measurements are taken increments until analyte onset is detected. In some embodiments, operation 715 is performed by a processor component that is the same as or similar to stage-two iterative processing component 34 (shown in FIG. 1 and described herein).

At an operation 720, the system may determine a first surrogate function or value based on the one or more first surrogate measurements and a second surrogate function or value based on at least one of the one or more second surrogate measurements. As an example, the first time period may be a period in which the measurements do not ramp (e.g., rise or fall sharply), and the second time period may be a period in which the measurements do ramp. The first and second surrogate values may each be a calculation (e.g., an average, median, mode, or other suitable, representative value) with respect to the measurements taken during the first and second time periods, respectively.

The first and second surrogate functions may be, if there are at least two data measurement points taken in the first and second time periods, respectively, a linear representation of the first and second time period measurements, respectively. For example, the first time period measurements may hover around the first surrogate value or function. Said hovering may be limited by a threshold value. That is, if a measurement presumably taken during a first time period exceeds the threshold value, then the measurement may actually be considered part of the second time period. As such, the second surrogate value or function may be relatively displaced from the first surrogate value or function by an amount that exceeds the same threshold value or another threshold value. As mentioned, the second surrogate function may be linear, if at least two data points have been taken in the second time period, but it may also be polynomial (e.g., parabolic), if at least three data points have been taken in the second time period.

Determining the first surrogate function or value, as part of operation 720, may include determining the first surrogate function or value based on the one or more first measurements of energy. Similarly, determining the second surrogate function or value may include determining the second surrogate function or value based on the one or more second measurements of energy. In some use cases, the one or more first surrogate measurements may include a plurality of first surrogate measurements. From those measurements, the system may determine a standard deviation of the plurality of first surrogate measurements. The system may, in some implementations, further determine, based on the standard deviation, the threshold as a threshold for analyte onset prediction specific to the individual under observation. The system may, in some implementations, compare the first and second surrogate functions against the plurality of first and second measurements, respectively, by curve-fitting the functions to the measurements. In some embodiments, operation 720 is, respectively with regard to the time period, performed by processor components that are the same as or similar to stage-one iterative processing component 32 and stage-two iterative processing component 34 (shown in FIG. 1 and described herein).

At an operation 725, the system may predict an onset of the sleep-related analyte for the first individual based on the first surrogate function or value and the second surrogate function or value such that the prediction of the onset of the sleep-related analyte is performed without determining any absolute concentration values for the sleep-related analyte. As an example, the system may determine a relative difference between the first surrogate function or value and the second surrogate function or value. Further, the system may determine whether the relative difference satisfies a threshold for analyte onset prediction. That is, in some examples, predicting the onset of the sleep-related analyte may include predicting the onset of the sleep-related analyte that is responsive to a determination that the relative difference satisfies the threshold for analyte onset prediction. As such, the prediction of the onset of the sleep-related analyte may be performed without determining an absolute concentration value for the sleep-related analyte. In some embodiments, operation 725 is performed by a processor component that is the same as or similar to analyte onset determination component 36 (shown in FIG. 1 and described herein).

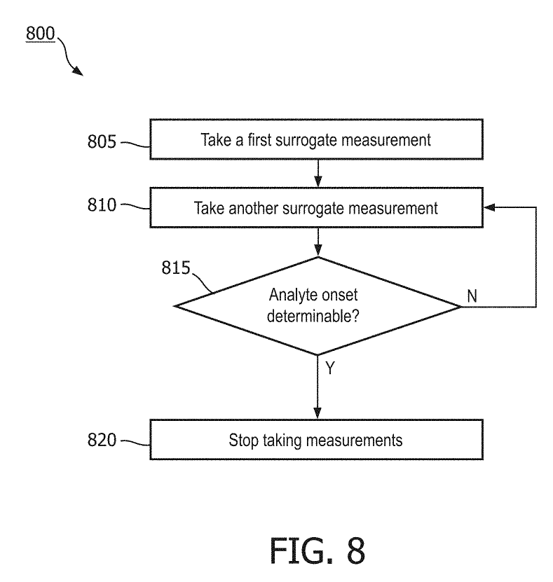

FIG. 8 illustrates a method 800 for determining an analyte's onset. Method 800 may be performed with an analyte onset determination system (e.g., system 10). The system comprises one or more hardware processors and/or other components. The hardware processors are configured by machine-readable instructions to execute computer program components. The computer program may include the same or similar components used to perform method 700. The operations of method 800 presented below are intended to be illustrative. In some embodiments, method 800 may be accomplished with one or more additional operations not described, and/or without one or more of the operations discussed. Additionally, the order in which the operations of method 800 are illustrated in FIG. 3 and described below is not intended to be limiting.

In some embodiments, method 800 may be implemented in one or more processing devices. The processing devices may include one or more devices executing some or all of the operations of method 800 in response to instructions stored electronically on an electronic storage medium. The processing devices may include one or more devices configured through hardware, firmware, and/or software to be specifically designed for execution of one or more of the operations of method 800.

At an operation 805, the system may take a first surrogate measurement. This measurement may be taken using the same or similar approach described with reference to operations 705 and 710. In some embodiments, operation 805 is performed by a processor component that is the same as or similar to sensor interface component 30 (shown in FIG. 1 and described herein).

At an operation 810, the system may take an additional surrogate measurement. This subsequent measurement may be taken using the same or similar approach described with reference to operation 705 and to operation 710 or 715. In some embodiments, operation 810 is performed by a processor component that is the same as or similar to stage-one iterative processing component 32 and/or stage-one iterative processing component 34 (shown in FIG. 1 and described herein).

At an operation 815, the system may determine whether the measured analyte has an observable onset. The onset may be determined via any one or more suitable method, such as by visual inspection, threshold-based (e.g., as discussed hereinafter with regard to method 900), curve-fitting (e.g., as discussed hereinafter with regard to method 1000), or differential equation based methods. If at operation 815 the system cannot determine the onset with the current amount of measurements taken, then the system may indefinitely return to executing operations 810 followed by 815. In some embodiments, operation 815 is performed by a processor component the same as or similar to analyte onset determination component 36 (shown in FIG. 1 and described herein).

At an operation 820, the system may stop taking measurements. That is, when the analyte's onset may be determined (e.g., with a relatively high degree of probability) the system need not continue to take measurements.

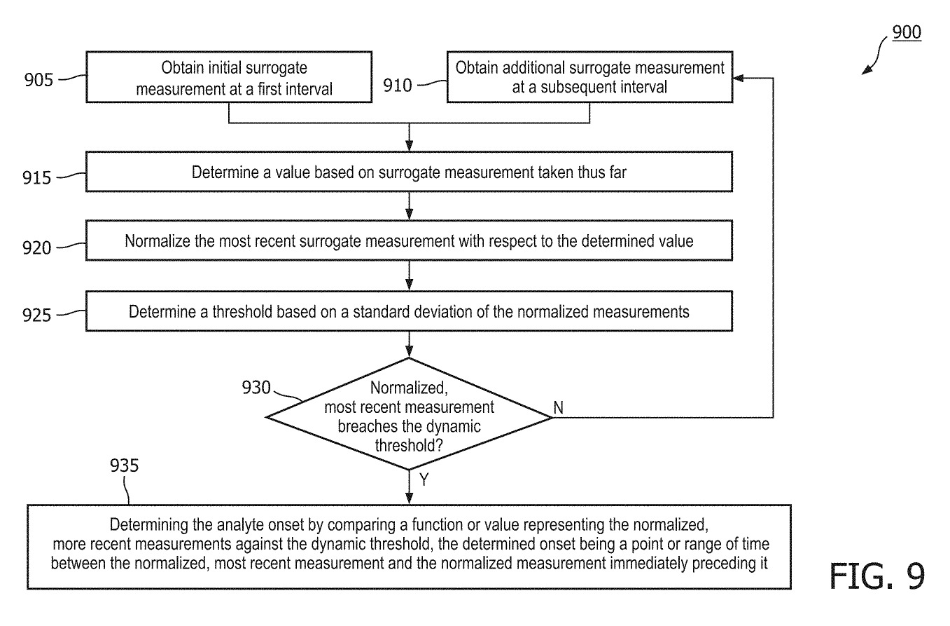

FIG. 9 illustrates a method 900 for determining an analyte's onset using a dynamic threshold. Method 900 may be performed with an analyte onset determination system (e.g., system 10). The system comprises one or more hardware processors and/or other components. The hardware processors are configured by machine-readable instructions to execute computer program components. The computer program may include the same or similar components used to perform method 700. The operations of method 900 presented below are intended to be illustrative. In some embodiments, method 900 may be accomplished with one or more additional operations not described, and/or without one or more of the operations discussed. Additionally, the order in which the operations of method 900 are illustrated in FIG. 3 and described below is not intended to be limiting.

In some embodiments, method 900 may be implemented in one or more processing devices. The processing devices may include one or more devices executing some or all of the operations of method 900 in response to instructions stored electronically on an electronic storage medium. The processing devices may include one or more devices configured through hardware, firmware, and/or software to be specifically designed for execution of one or more of the operations of method 900.

At an operation 905, the system may obtain an initial surrogate measurement at the beginning of a first interval. This measurement may be taken using the same or similar approach described with reference to operations 705 and 710. In some embodiments, operation 905 is performed by a processor component that is the same as or similar to sensor interface component 30 (shown in FIG. 1 and described herein).

At an operation 915, the system may determine a value based on surrogate measurements taken thus far. As an example, the baseline value may be an average, median, or mode of the current measurements (e.g., measured as an amount of energy detected or sensed as reflected in a measurement device), which have little or no analyte in the measured samples. In some implementations, the baseline value “B0” may be based off a precision or noise level in the device itself or based off a measurement taken without a sample. In other implementations, the baseline value may be predetermined or determined, e.g., as batch calibration data, precalibrated coefficient of variation data, user-configurable via a processor component that is the same as or similar to user interface component 38, based on measurements prior to the current time period of observation, or based on another parameter. In some embodiments, operation 915 is performed by a processor component that is the same as or similar to stage-one iterative processing component 32 (shown in FIG. 1 and described herein).

At an operation 920, the system may normalize the most recent surrogate measurement with respect to the baseline value determined in operation 915. In one use case, the measurement (e.g., the data point “B” of operation 905 or 910) may be divided by the determined baseline value. In some use cases, the measurement or quotient is further converted to determine a signal change S, which may be equal to 100−B (where S is a percentage value) or 1−B/B0. In some instances, S and/or B may be a decimal value and in others a percentage value. The measurement “B” may be converted to signal “S” so that a rise (or fall) in an analyte's measurement leads to a rise (or fall) in the signal. In some embodiments, operation 920 is performed by a processor component that is the same as or similar to stage-one iterative processing component 32 (shown in FIG. 1 and described herein).

At an operation 925, the system may determine a dynamic threshold in any number of ways. For example, it may be based on a characteristic of the measurements (e.g., on a standard deviation of the normalized measurements) taken thus far. The system may alternatively operate with a dynamic threshold that is static, e.g., predetermined, user-configurable via a processor component that is the same as or similar to user interface component 38, or based on another parameter. In some implementations, the dynamic threshold may be larger than the baseline value by, e.g., the standard deviation, double the standard deviation, triple the standard deviation, quadruple the standard deviation, quintuple the standard deviation, any multiple of it, or based on another parameter. In some embodiments, operation 925 is performed by a processor component that is the same as or similar to stage-one iterative processing component 32 (shown in FIG. 1 and described herein).

At an operation 910, the system may obtain an additional surrogate measurement at a subsequent interval. As an example, this measurement may be taken using the same or similar approach described with reference to operations 705 and 710. Operation 910 may be executed, in some embodiments, if the measurement analyzed at operation 930 fails to breach the dynamic threshold. In some embodiments, operation 910 is performed by a processor component that is the same as or similar to sensor interface component 30 (shown in FIG. 1 and described herein).

At an operation 930, the system may determine whether the normalized, most recent measurement breaches the dynamic threshold. For example, the system may compare the normalized, most recent measurement to determine whether it satisfies or breaches the dynamic threshold. In some embodiments, operation 930 is performed by a processor component the same as or similar to stage-two iterative processing component 34 (shown in FIG. 1 and described herein).

At an operation 935, the system may determine the analyte's onset by comparing a function or value representing the normalized, more recent measurements against the dynamic threshold. The determined onset may be a point of time or a range of time between times at which the most recent and penultimate measurements were taken. For example, as shown in FIG. 5, the determined analyte onset may be a time at which an imaginary line, drawn at the dynamic threshold level determined as part of operation 925, intersects with a function or value representing the ramping portion of the normalized measurements. In some embodiments, operation 935 is performed by a processor component the same as or similar to analyte onset determination component 36 (shown in FIG. 1 and described herein).

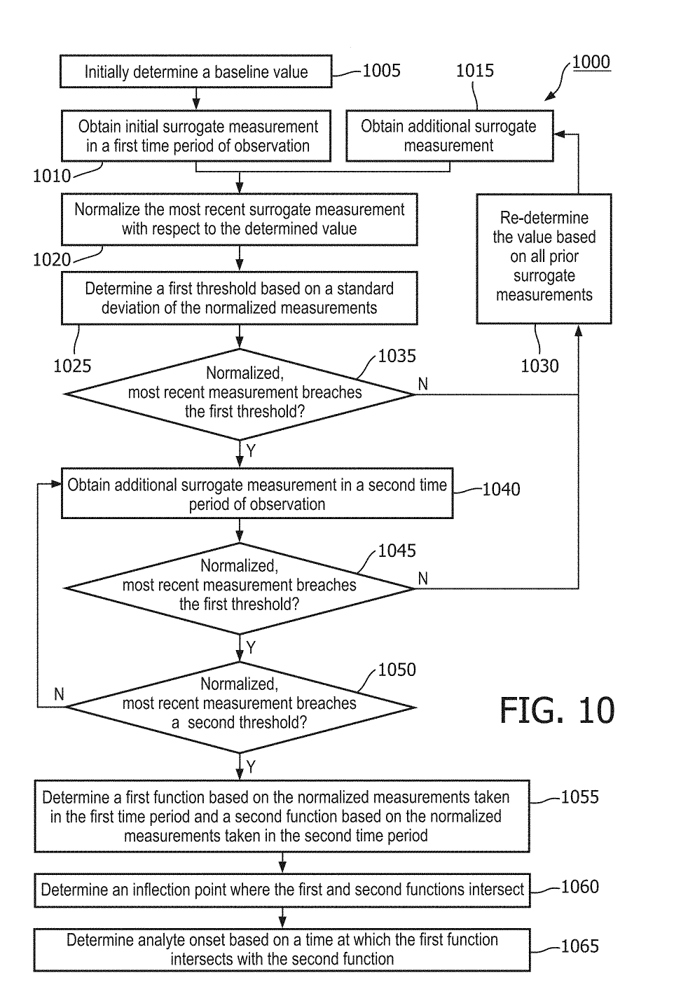

FIG. 10 illustrates a method 1000 for determining an analyte's onset using an adapted hockey-stick method. Method 1000 may be performed with an analyte onset determination system (e.g., system 10). The system comprises one or more hardware processors and/or other components. The hardware processors are configured by machine-readable instructions to execute computer program components. The computer program may include the same or similar components used to perform method 700. The operations of method 1000 presented below are intended to be illustrative. In some embodiments, method 1000 may be accomplished with one or more additional operations not described, and/or without one or more of the operations discussed. Additionally, the order in which the operations of method 1000 are illustrated in FIG. 10 and described below is not intended to be limiting.

In some embodiments, method 1000 may be implemented in one or more processing devices. The processing devices may include one or more devices executing some or all of the operations of method 1000 in response to instructions stored electronically on an electronic storage medium. The processing devices may include one or more devices configured through hardware, firmware, and/or software to be specifically designed for execution of one or more of the operations of method 1000.

At an operation 1005, the system may initially determine a baseline value. As an example, the baseline value may be an amount of energy detected (or sensed) to be reflected in a measurement device (e.g., measurement device 18) that has little or no analyte in the measured sample. In some implementations, the baseline value “B0” may be based off a precision or noise level in the device itself or based off a measurement taken without a sample. In other implementations, the baseline value may be predetermined or determined, e.g., as batch calibration data, precalibrated coefficient of variation data, user-configurable via a processor component that is the same as or similar to user interface component 38, based on measurements prior to the current time period of observation, or based on another parameter. In some embodiments, operation 1005 is performed by a processor component that is the same as or similar to sensor interface component 30 (shown in FIG. 1 and described herein).

At an operation 1010, the system may obtain an initial measurement, the initial measurement being taken in a first time period of observation that begins with this measurement. This measurement may be taken using the same or similar approach described with reference to operations 705 and 710. In some embodiments, operation 1010 is performed by a processor component that is the same as or similar to sensor interface component 30 (shown in FIG. 1 and described herein).

At an operation 1020, the system may normalize the most recent surrogate measurement with respect to the value determined in operation 1005. In some implementations, the measurement may be normalized via a same or similar approach used as part of method 900, specifically operation 920. In some embodiments, operation 1020 is performed by a processor component that is the same as or similar to stage-one iterative processing component 32 (shown in FIG. 1 and described herein).

At an operation 1025, the system may determine a first threshold in any number of ways. For example, it may be based on the approach introduced with respect to operation 925. In some embodiments, operation 1025 is performed by a processor component that is the same as or similar to stage-one iterative processing component 32 (shown in FIG. 1 and described herein).

At an operation 1035, the system may determine whether to take additional measurements as part of the first time period of observation. For example, the system may compare the normalized, most recent measurement to determine whether it satisfies or breaches the first threshold. In some embodiments, operation 1035 is performed by a processor component that is the same as or similar to stage-one iterative processing component 32 (shown in FIG. 1 and described herein).

At an operation 1030, the system may re-determine the baseline value determined in operation 1005. That is, the value may be determined based on all prior surrogate measurements in the first time period of observation of the individual. For example, the prior measurements may be averaged (mean) or the mode, median, or other relative value may be used as the baseline value B0. The baseline analysis may be developed and optimized to, e.g., narrow down the lower boundary for the analyte's onset. As long as no significant rise in the signal is observed, data measurement points may be allocated to the baseline portion of the signal profile. In some embodiments, operation 1030 is performed by a processor component that is the same as or similar to stage-one iterative processing component 32 (shown in FIG. 1 and described herein).

At an operation 1015, the system may obtain an additional surrogate measurement and operations 1020, 1025, and 1035 may indefinitely execute in order until the first threshold is breached. In some embodiments, operation 1015 is performed by a processor component that is the same as or similar to sensor interface component 30 (shown in FIG. 1 and described herein).

At an operation 1040, the system may obtain an additional surrogate measurement in a second time period of observation. At this point, all prior measurements may be deemed to have been taken in the first time period of observation, and the most recent measurement may be deemed to have been taken in the second time period of observation. In some use cases, the second time period of observation may be when the sampled measurements begin to ramp, rise, or fall relative to the baseline portion. In some embodiments, operation 1040 is performed by a processor component that is the same as or similar to sensor interface component 30 (shown in FIG. 1 and described herein).

At an operation 1045, the normalized, most recent measurement may be checked to determine whether it breaches the first threshold. In most use cases, this will be true (Y, as in “yes”) and the system will advance to executing operation 1050. In other use cases, this will be false (N, as in “no”), the penultimate measurement being a false-positive for identifying a significant ramp of measurements. Accordingly, in these other use cases the system may return to operation 1030, for re-determining the baseline value, since the first time period of observation is deemed to be continuing. In some embodiments, operation 1045 is performed by a processor component the same as or similar to stage-two iterative processing component 34 (shown in FIG. 1 and described herein).

At an operation 1050, assuming that the (e.g., normalized) most recent measurement breaches the first threshold then the measurement is checked to determine whether it also breaches a second threshold. As an example, the second threshold may be based on a characteristic of the measurements taken thus far (e.g., a standard deviation of the normalized measurements) or, alternatively, the second threshold may be static, e.g., predetermined, user-configurable via a processor component that is the same as or similar to user interface component 38, or based on another parameter. In some implementations, the second threshold may be beyond the baseline value by, e.g., double the standard deviation, triple it, quadruple it, quintuple it, any multiple of it, or based on another parameter. In some implementations, the second threshold is a larger (e.g., more positive if rising or more negative if falling) value than the first threshold.

While the first threshold may be set to a small value to ensure that some (e.g., several) data points are taken for estimating the analyte's onset, the second threshold determination may allow the narrowing down of options from a possible range for onset determination based on an increased probability that a larger second threshold results in a higher signal measurement that corresponds to the ramping portion of the signal profile. The resulting, possible range may thus be large with these lower and upper bounds for determining the onset, resulting in a high probability of successfully determining the onset. By comparing against the second threshold, the system may determine (e.g., in the event that the normalized, most recent measurement breaches the second threshold) that an onset had occurred during that sampling period. In some embodiments, operation 1050 is performed by a processor component the same as or similar to stage-two iterative processing component 34 (shown in FIG. 1 and described herein).

At an operation 1055, the system may determine a first function based on the normalized measurements taken in the first time period and a second function based on the normalized measurements taken in the second time period. In some embodiments, the first function may be determined at an earlier operation, e.g., when determining whether to start taking additional measurements as part of the second time period of observation. As an example, the first function may be linear, e.g., a flat line or line with constant slope. As an example, the second function may be linear or polynomial (e.g., parabolic). In some examples, an initial portion of the second function may change at a faster rate (e.g., have a greater slope) than the first function. In some embodiments, operation 1055 is performed by a processor component the same as or similar to stage-two iterative processing component 34 (shown in FIG. 1 and described herein).

At an operation 1060, the system may determine an inflection point where the first and second functions intersect. In some embodiments, operation 1060 is performed by a processor component the same as or similar to analyte onset determination component 36 (shown in FIG. 1 and described herein).

At an operation 1065, the system may determine analyte onset based on a time at which the first function intersects with the second function. As an example, the onset time may be determined relative to or at a time where the flat line of FIG. 6 intersects with the curved line that follows it. That is, in this example, the onset time may be deemed 20:57 hours, which is just before the 21:00 measurement. In another example, the onset time may be deemed just after the 22:00 measurement, as shown in FIG. 5. Upon determining the analyte's onset, subsequent measurements (e.g., showing a saturation in the analyte) are unnecessary and, in some embodiments, not taken. In some embodiments, operation 1065 is performed by a processor component the same as or similar to analyte onset determination component 36 (shown in FIG. 1 and described herein).

Although the description provided above provides detail for the purpose of illustration based on what is currently considered to be the most practical and preferred embodiments, it is to be understood that such detail is solely for that purpose and that the disclosure is not limited to the expressly disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

What is claimed is:

1. A method for determining onset of a sleep-related analyte, the method being implemented by one or more processors configured by machine-readable instructions, the method comprising:
obtaining one or more first surrogate measurements of a sleep-related analyte based on one or more signals obtained via one or more sensors during a first time period of observation of a first individual;
obtaining one or more second surrogate measurements of the sleep-related analyte based on one or more signals obtained via one or more sensors during a second time period of observation of the first individual;
determining a first surrogate function or value based on the one or more first surrogate measurements and a second surrogate function or value based on the one or more second surrogate measurements; and
predicting an onset of the sleep-related analyte for the first individual based on the first surrogate function or value and the second surrogate function or value such that the prediction of the onset of the sleep-related analyte is performed without the one or more processors determining an absolute concentration value for the sleep-related analyte.

2. The method of claim 1, wherein the one or more first surrogate measurements comprise one or more first measurements of energy obtained via one or more sensors during the first time period, and the one or more second surrogate measurements comprise one or more second measurements of energy obtained via one or more sensors during the second time period, and
wherein determining the first surrogate function or value comprises determining the first surrogate function or value based on the one or more first measurements of energy, and
wherein determining the second surrogate function or value comprises determining the second surrogate function or value based on the one or more second measurements of energy.

3. The method of claim 2, wherein the one or more first measurements of energy comprises one or more first measurements of light obtained via one or more sensors during the first time period, and the one or more second measurements of energy comprises one or more second measurements of light obtained via one or more sensors during the second time period, and wherein determining the first surrogate function or value comprises determining the first surrogate function or value based on the one or more first measurements of light, and wherein determining the second surrogate function or value comprises determining the second surrogate function or value based on the one or more second measurements of light.

4. The method of claim 1, further comprising:
determining a relative difference between the first surrogate function or value and the second surrogate function or value; and
determining whether the relative difference satisfies a first threshold for analyte onset prediction, wherein predicting the onset of the sleep-related analyte comprises predicting the onset of the sleep-related analyte, responsive to a determination that the relative difference satisfies the first threshold for analyte onset prediction, such that the prediction of the onset of the sleep-related analyte is performed without determining an absolute concentration value for the sleep-related analyte.

5. The method of claim 4, wherein the one or more first surrogate measurements comprises a plurality of first surrogate measurements, the method further comprising:
determining a standard deviation of the plurality of first surrogate measurements; and
determining, based on the standard deviation, the first threshold as a threshold for analyte onset prediction specific to the first individual.

6. The method of claim 1, wherein the first surrogate function or value comprises a first surrogate function, and the second surrogate function or value comprises a second surrogate function, and wherein at least an initial portion of the second surrogate function changes value over time at a greater rate than the first surrogate function.

7. The method of claim 1, wherein the first surrogate function or value comprises a first surrogate function, and the second surrogate function or value comprises a second surrogate function, and wherein the onset is predicted based on a time at which the first surrogate function intersects with the second surrogate function.

8. The method of claim 1, wherein the first and second surrogate measurements are obtained in vitro via samples of blood, urine, or saliva of the first individual.

9. The method of claim 1, wherein the first surrogate function or value comprises a first surrogate function, and the second surrogate function or value comprises a second surrogate function, and wherein the first surrogate function is linear, and wherein the second surrogate function is a polynomial curve.

10. The method of claim 1, wherein each of the one or more first surrogate measurements and the one or more second surrogate measurements is obtained via an immunoassay based on magnetic colloids or beads.

11. A system configured to determine onset of a sleep-related analyte, the system comprising one or more processors configured by machine-readable instructions to:
obtain one or more first surrogate measurements of a sleep-related analyte based on one or more signals obtained via one or more sensors during a first time period of observation of a first individual;
obtain one or more second surrogate measurements of the sleep-related analyte based on one or more signals obtained via one or more sensors during a second time period of observation of the first individual;
determine a first surrogate function or value based on the one or more first surrogate measurements and a second surrogate function or value based on the one or more second surrogate measurements; and
predict an onset of the sleep-related analyte for the first individual based on the first surrogate function or value and the second surrogate function or value such that the prediction of the onset of the sleep-related analyte is performed without the one or more processors determining an absolute concentration value for the sleep-related analyte.

12. The system according to claim 11, wherein the one or more first surrogate measurements comprise one or more first measurements of energy obtained via one or more sensors during the first time period, and the one or more second surrogate measurements comprise one or more second measurements of energy obtained via one or more sensors during the second time period, and wherein determining the first surrogate function or value comprises determining the first surrogate function or value based on the one or more first measurements of energy, and wherein determining the second surrogate function or value comprises determining the second surrogate function or value based on the one or more second measurements of energy.

13. The system according to claim 11, wherein the one or more first measurements of energy comprises one or more first measurements of light obtained via one or more sensors during the first time period, and the one or more second measurements of energy comprises one or more second measurements of light obtained via one or more sensors during the second time period, and wherein determining the first surrogate function or value comprises determining the first surrogate function or value based on the one or more first measurements of light, and wherein determining the second surrogate function or value comprises determining the second surrogate function or value based on the one or more second measurements of light.

14. The system according to claim 11, wherein the one or more processors are further configured to:
determine a relative difference between the first surrogate function or value and the second surrogate function or value; and
determine whether the relative difference satisfies a first threshold for analyte onset prediction, wherein predicting the onset of the sleep-related analyte comprises predicting the onset of the sleep-related analyte, responsive to a determination that the relative difference satisfies the first threshold for analyte onset prediction, such that the prediction of the onset of the sleep-related analyte is performed without determining an absolute concentration value for the sleep-related analyte.

15. The system according to claim 11, wherein the one or more first surrogate measurements comprises a plurality of first surrogate measurements, and wherein the one or more processors are further configured to:
determine a standard deviation of the plurality of first surrogate measurements; and
determine, based on the standard deviation, the first threshold as a threshold for analyte onset prediction specific to the first individual.

16. The system according to claim 11, wherein each of the one or more first surrogate measurements and the one or more second surrogate measurements is obtained via an immunoassay based on magnetic colloids or beads.

17. A system for determine onset of a sleep-related analyte, the system comprising:
means for obtaining one or more first surrogate measurements of a sleep-related analyte based on one or more signals obtained via one or more sensors during a first time period of observation of a first individual;
means for obtaining one or more second surrogate measurements of the sleep-related analyte based on one or more signals obtained via one or more sensors during a second time period of observation of the first individual;
means for determining a first surrogate function or value based on the one or more first surrogate measurements and a second surrogate function or value based on the one or more second surrogate measurements; and
means for predicting an onset of the sleep-related analyte for the first individual based on the first surrogate function or value and the second surrogate function or value such that the prediction of the onset of the sleep-related analyte is performed without determining an absolute concentration value for the sleep-related analyte.

18. The system according to claim 17, wherein the one or more first surrogate measurements comprise one or more first measurements of energy obtained via one or more sensors during the first time period, and the one or more second surrogate measurements comprise one or more second measurements of energy obtained via one or more sensors during the second time period, and wherein determining the first surrogate function or value comprises determining the first surrogate function or value based on the one or more first measurements of energy, and wherein determining the second surrogate function or value comprises determining the second surrogate function or value based on the one or more second measurements of energy.

19. The system according to claim 17, wherein the one or more first measurements of energy comprises one or more first measurements of light obtained via one or more sensors during the first time period, and the one or more second measurements of energy comprises one or more second measurements of light obtained via one or more sensors during the second time period, and wherein determining the first surrogate function or value comprises determining the first surrogate function or value based on the one or more first measurements of light, and wherein determining the second surrogate function or value comprises determining the second surrogate function or value based on the one or more second measurements of light.

20. The system according to claim 17, further comprising:
means for determining a relative difference between the first surrogate function or value and the second surrogate function or value; and
means for determining whether the relative difference satisfies a first threshold for analyte onset prediction, wherein predicting the onset of the sleep-related analyte comprises predicting the onset of the sleep-related analyte, responsive to a determination that the relative difference satisfies the first threshold for analyte onset prediction, such that the prediction of the onset of the sleep-related analyte is performed without determining an absolute concentration value for the sleep-related analyte.
